(19) United States
(12) Patent Application Publication
(10) Pub. No.: US 2003/0064405 A1
(43) Pub. Date: Apr. 3, 2003

(54) Combined device for the treatment of biological micro-networks

(76) Inventors: Hans Sigrist, Kernenried (CH); Friedrich Heitger, Berne (CH); Cedric Faure, Cortaillod (CH)

Correspondence Address: VAN TASSEL AND ASSOCIATES, POST OFFICE BOX 2928, BELLAIRE, TX, 77402-2928, US

(21) Appl. No.: 10/262,243

(22) Filed: Oct. 1, 2002

(30) Foreign Application Priority Data
Oct. 3, 2001 (EP) O1 810 961.1

(51) Int. Cl. C12Q 1/68; B41J 2/015; C12M 1/34

(52) U.S. Cl. 435/6; 435/287.2; 347/20

(57) ABSTRACT

The invention concerns a device for the treatment, transport and/or storage of a substrate plate (10) on which has been deposited at least one micro-network (12) of samples of compounds to be tested.

It is formed by superposing two plates, one lower (14), the other upper (16), intended to be attached to and detachable from the substrate plate. The lower plate (14) has an aperture (20) designed to be situated above the micro-network (12) to form a reaction chamber, sealed at the top by the upper plate (16). Means (28) are provided to introduce a reagent into the chamber, from the upper plate.

The present invention relates to the field of micro-networks of samples of biological, biochemical or chemical compounds. It concerns, more particularly, a combined packaging and testing device for such micro-networks.

Pharmaceutical chemistry and biochemistry, in particular, need to test the potential therapeutic activity of molecules by placing them in contact with specific reagents. Through appropriate processes of analysis, it is then possible to measure the effectiveness of the tested molecule.

At present, there are thousands of molecules that have to be tested in this way for each field of research. It is therefore essential to optimise the execution and cost of these experiments, bearing in mind that in view of the fineness of the analyses involved, it is inconceivable to risk the slightest outside contamination.

For such operations it is common to use substrate plates on which have been “printed”, by means of appropriate tools, micro-networks of samples of molecules, which must then be protected within packaging so that they can be transported and stored under optimum conditions with a view to subsequent testing in reaction chambers which, due to the high cost of the reagents used, must be miniaturized to the greatest possible extent.

The aim of the present invention is to provide a device by means of which the operations of storage, transport and placing in contact with reagents (analysis of molecular interactions and/or development) are greatly facilitated, at very low cost and without risk of contamination.

More precisely, the invention concerns a device for the treatment of a substrate plate on which has been deposited at least one micro-network of samples of compounds to be tested, characterized:

in that it is formed by the superposing of two plates, one lower, the other upper, intended to be attached to and detachable from the substrate plate, said plates being rigidly joined together to form a functional entity,

in that the lower plate has an aperture designed to be situated above said micro-network to form a reaction chamber that is sealed, at the top, by the upper plate, and

in that it contains means for introducing a reagent into the chamber, from the upper plate.

The device according to the invention benefits in addition from the following characteristics.

On the upper plate, adjacent to the aperture in the lower plate, there is an extra-thick portion the dimensions of which enable it to fit inside the aperture, without filling it completely, in order to reduce the height of the reaction chamber.

The height of the chamber is between 0.03 and 0.8 mm.

The aperture in the lower plate has a surface area of between 0.1 and 8 cm².

The introducing means comprises two connectors formed on the top of the upper plate, each of which communicates with a groove made in its underside and ending in the reaction chamber, said grooves forming, with the upper surface of the lower plate, micro-channels, one of which is used to inject the reagent into the chamber, the other to extract it.

The micro-channels have a cross-section, preferably, of between 500 and 12,000 μm².

The lower and upper plates are fused or bonded together.

The device is attached to the substrate plate by means of clips.

The device contains means to hermetically seal the reaction chamber.

The underside of the lower plate has a deposit of weak adhesive around its aperture.

The lower and upper plates have dimensions roughly equal to those of the substrate plate.

The lower and upper plates are made of a material chosen for its physico-chemical properties or its compatibility with the compounds to be tested.

The surfaces intended to come into contact with the compounds to be tested have previously been passivated, in order to reduce their capacity to adsorb the compounds to be tested.

A better understanding of the invention can be gained by reading the description that follows, made with reference to the annexed drawing on which:

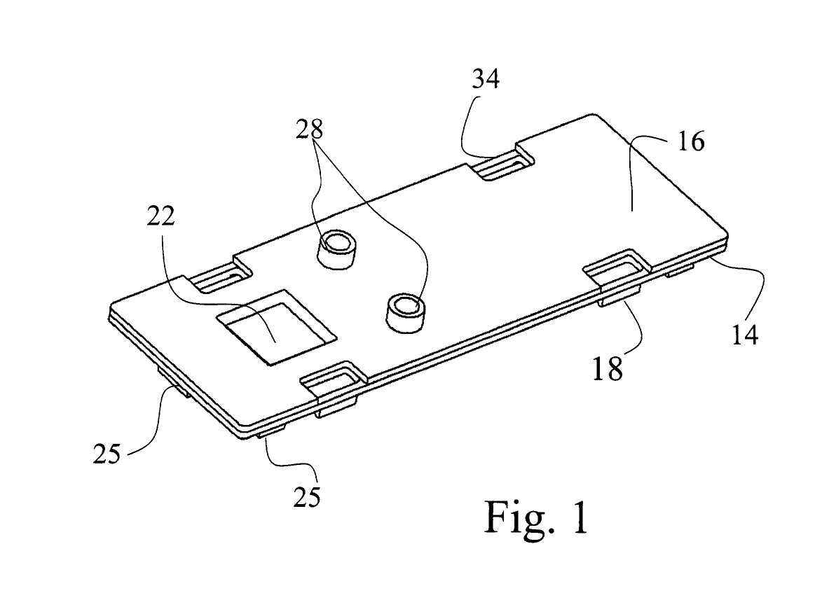

FIG. 1 is a representation in three dimensions of the device according to the invention;

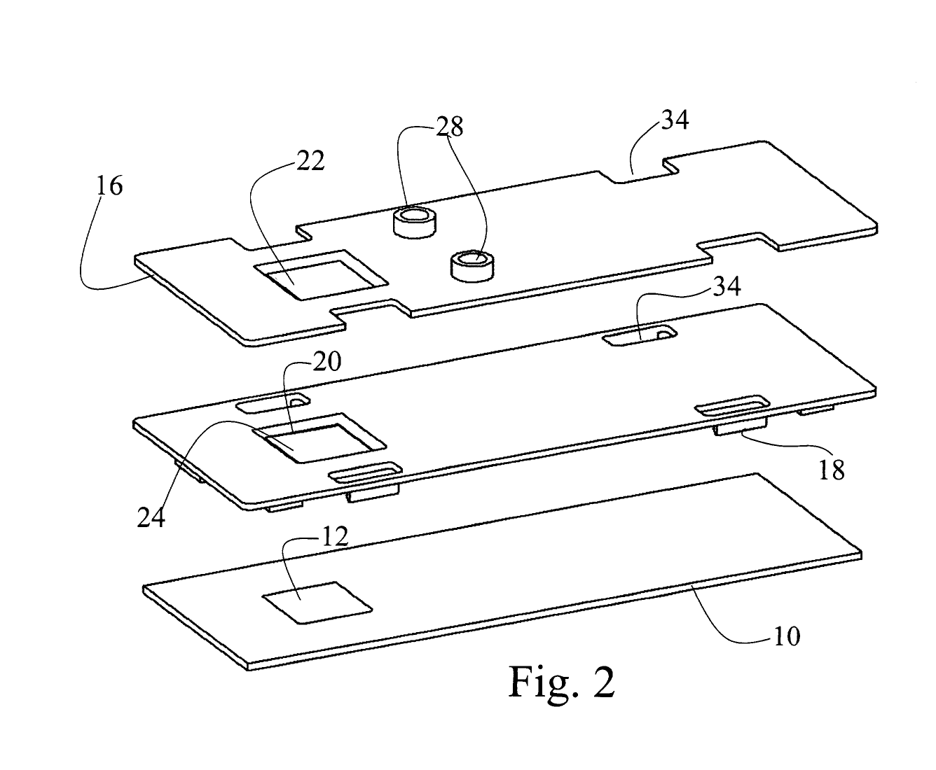

FIG. 2 is an exploded view in three dimensions of the device;

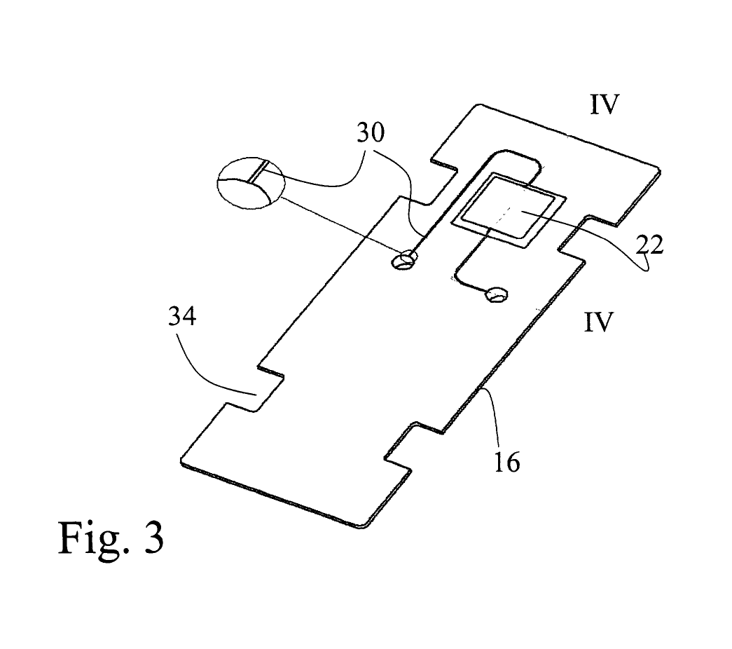

FIG. 3 is a horizontal projection of the upper plate;

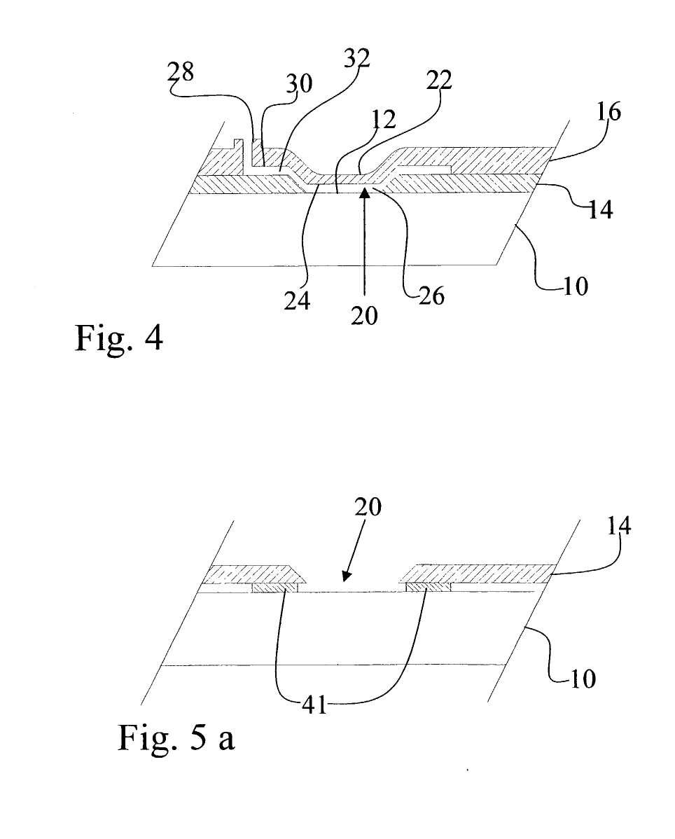

FIG. 4 is a partial cross-section of the device according to IV-IV, and

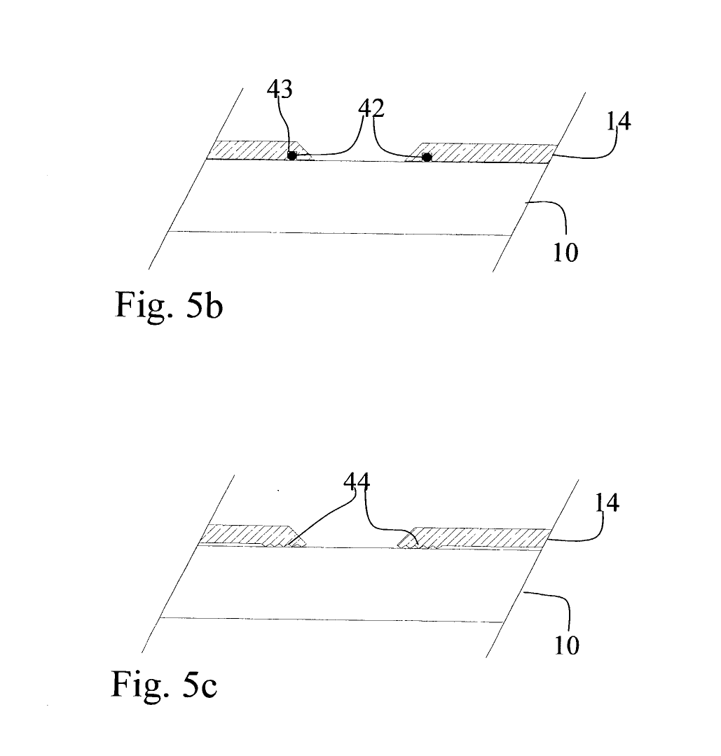

FIG. 5 shows several variants of the means to hermetically seal the reaction chamber.

On these figures, reference 10 shows a substrate plate of standard dimensions, typically 25×75×1.1 mm according to the metric system, along the longitudinal axis of which has been affixed beforehand, using well known techniques, a micro-network 12 of samples of biological compounds that may be, in particular, biomolecules (nucleic acids, oligonucleotides, proteins, enzymes, antibodies, allergens, peptides, lectins, receptors, carbohydrates, oligo- or polysaccharides, lipids, compounds of low molecular weight, etc), assemblages of such biomolecules, of viruses, of cells, of bacteria, of tissues, etc. For information, the micro-network 12 has a length of 3 to 40 mm and a width of 3 to 20 mm. It may, according to applications, contain a single homogeneous surface of biological compounds or a plurality of such surfaces distributed in a network.

The device according to the invention is intended to form a detachable annex to the plate 10 in order to protect the micro-network 12, allow its transportation and storage, and then serve as a vessel in which its samples can react with an appropriate reagent.

As can be seen from the figures, the device according to the invention comprises a lower plate 14 and an upper plate 16 which are joined together, rectangular, and of dimensions similar to those of the substrate 10. They are approximately 0.5 to 2 mm thick and are both made from a rigid or flexible material chosen for its physico-chemical properties or its compatibility with the compounds to be tested. Advantageously the material used may be, for example, silicon, an organic polymer such as polymethyl methacrylate, a polycarbonate, a multi-component polymer, etc.

The contour of the lower plate 14 is fitted with a number of downward facing hooks 18, intended to cooperate with the circumference of the plate 10 to form a reversible join between the two plates by means of clips.

In addition, the lower plate 14 has an aperture 20 of unspecified shape along its longitudinal axis, the surface area of which is typically between 0.25 and 5 cm² and the edges of which are bevelled. The aperture 20 is arranged and dimensioned such that it is located precisely above the micro-network 12 when the device is placed on the substrate 10.

The upper plate 16 exhibits, adjacent to the aperture 20 of the lower plate, an indentation 22 of which the base 24 protrudes beyond its lower surface and is dimensioned so that it fits into the aperture 20, without filling it completely, to form a reaction chamber 26 directly above the network 12. Typically, the height of this chamber, determined by the depth of the indentation 22, may be between 0.05 and 0.8 mm.

The upper surface of the plate 16 exhibits, in addition, two tubular connectors 28 each communicating with a groove 30 made in the underside of the plate and ending in the chamber 26, at opposite ends of the base 24 of the indentation 22. These grooves 30 have a cross-section of between 500 and 12,000 μm² and are intended to form micro-channels 32 at the interface of the plates 14 and 16 when the latter are assembled. The connectors 28 are dimensioned such that one is used to inject a liquid reagent into the chamber 26 and the other to extract it, for example by means of syringes.

The two plates 14 and 16 are fused or bonded together, or joined permanently by any other technique. It will be noted that each has opposing lateral notches 34 intended for manipulation by robots. In addition, the plate 14 preferably contains lugs 25 which, situated around its perimeter, ensure accurate positioning of the device on the substrate plate 10.

Lastly, the lower surface of the plate 14 may be given a deposit of weak adhesive (not shown on the drawings) around the aperture 20, plus a protective film of removable plastic (also not shown) which covers at least the adhesive and the aperture. The reaction chamber 26 is thus confined and kept clean, independently of the presence of the substrate plate 10. Several devices such as the one described can thus be stacked, pending use, without any risk of contamination.

It is particularly advantageous if the surfaces of the device intended to come into contact with the biological compounds to be tested are bio-passivated, in other words treated, in accordance with procedures well known to specialists in the field, using products that remove their capacity to adsorb bio-molecules. Preferably, such passivation is carried out using photo-activated polysaccharides.

It is also advantageous if the upper plate 16, at least, is transparent, thereby enabling movement of the reagent in the device to be monitored visually.

The device according to the invention must also contain means enabling the reaction chamber to be hermetically sealed when it is clipped to the substrate plate 10. Such means may consist of an adhesive 41 (FIG. 5.a), preferably reversible to allow disassembly of the plate 10, placed on the underside of the plate 14 around the aperture 20. They may also consist of an O-ring seal 42 placed in a groove 43 provided for this purpose (FIG. 5.b). Lastly, according to an advantageous variant (FIG. 5.c), such means may consist of local concentric microstructures 44 on the underside of the plate 14. These microstructures are such that they lose their shape under the action of the pressure exerted by the device when it is clipped to the substrate plate. Typically, these microstructures may have a height of 10 μm and a spacing of 2 to 5 μm. The benefit of this variant is that it can be achieved by the same production method as the plate 14 itself, for example by injection moulding, thereby requiring no additional components or manufacturing stages.

Use of the device according to the invention is very simple. Once the substrate plate 10 has received its micro-network 12 of biomolecules to be tested, the operator, when necessary, simply removes the protective plastic film covering the adhesive on the underside of the plate 14 and clips the device to the plate 10. The reaction chamber 26 is thus situated over the micro-network 12, with the seal around the aperture 20 ensuring that the reaction chamber is hermetically sealed.

The entire assembly may then be stored and transported easily and safely.

Subsequently, when a test is required to be carried out, an appropriate reagent is injected into one of the connectors 28 and circulates through the corresponding micro-channel 32 as far as the chamber 26, where it reacts with the samples of biomolecules of the micro-network 12 before being evacuated through the other micro-channel and the other connector.

All that is then required is to detach the device from the substrate plate 10 to obtain a “developed” micro-network ready to be examined, for example optically, or to receive an appropriate surface treatment. The device may finally either be disposed of, or combined with another substrate plate after cleaning.

The present description refers to a substrate plate containing only a single micro-network of biological compounds, however it is clear that the device according to the invention can be used with networks containing a plurality (up to 10) of micro-networks, the latter able to be used simultaneously or sequentially. In this case, the device may be fitted with additional connectors and/or micro-channels allowing all necessary interconnections to be made.

It is clear, moreover, that the device may be attached to the substrate plate by any means ensuring a detachable assembly, for example by means of a clamp or a clip.

In this way a miniaturized device can be assembled which, serving both as packaging and as a reaction chamber for the micro-network, simplifies all handling operations, reduces the risk of contamination to a minimum and is very inexpensive to produce.

1. A device for the treatment of a substrate plate on which has been deposited at least one micro-network of samples of compounds to be tested, characterized:
in that it is formed by the superposing of two plates, one lower, the other upper, intended to be attached to and detachable from the substrate plate, said plates being rigidly joined together to form a functional entity,
in that the lower plate has an aperture designed to be located above said micro-network to form a reaction chamber that is sealed, at the top, by the upper plate, and
in that it comprises means for introducing a reagent into said chamber, from the upper plate.

2. The device of claim 1, containing, in addition, sealing means deployed around said aperture and intended to hermetically seal the reaction chamber when it is assembled on the substrate plate.

3. The device of claim 1, wherein the upper plate has, adjacent to the aperture in the lower plate, an extra-thick portion dimensioned so that it fits into said aperture, without filling it completely, in order to reduce the height of the reaction chamber.

4. The device of claim 3, wherein the height of said chamber is between 0.03 and 0.8 mm.

5. The device of claim 1, wherein said aperture has a surface area of between 0.1 and 8 cm².

6. The device of claim 1, wherein said introducing means comprises two connectors formed on the top of the upper plate, each communicating with a groove made in its lower surface and ending in the reaction chamber, said grooves forming, with the upper surface of the lower plate, micro-channels, one of which is used to inject said reagent into the chamber, the other to extract it.

7. The device of claim 6, wherein said micro-channels have a cross-section of between 500 and 12,000 μm².

8. The device of claim 1, wherein said lower and upper plates are fused or bonded together.

9. The device of claim 1, characterized in that it is attached to the substrate plate by means of clips.

10. The device of claim 1, wherein the underside of the lower plate contains a deposit of weak adhesive around its aperture.

11. The device of claim 1, wherein said lower and upper plates are made from a material chosen for its physico-chemical properties or its compatibility with the compounds to be tested.

12. The device of claim 1, wherein the surfaces intended to come into contact with the compounds to be tested have previously been passivated in order to reduce their capacity to adsorb said compounds.

13. The device of claim 2, wherein said sealing means consists of a reversible adhesive.

14. The device of claim 2, wherein said sealing means consists of a seal placed in a groove on the outer surface of the said lower plate.

15. The device of claim 2, wherein said sealing means consists of microstructures forming concentric and deformable excrescences on the outer surface of the said lower plate.
